(19) United States
(12) Patent Application Publication
(10) Pub. No.: US 2023/0007953 A1
(43) Pub. Date: Jan. 12, 2023

(54) DEVICE FOR CONTROLLING MOTION SICKNESS, WHICH IS INTEGRATED INTO A MOTOR VEHICLE

(71) Applicant: NOVARES FRANCE, CLAMART (FR)

(72) Inventors: Christophe CAZES, VERSAILLES (FR); François BOLLIER, NICE (FR)

(21) Appl. No.: 17/784,810

(22) PCT Filed: Nov. 18, 2020
(86) PCT No.: PCT/FR2020/052108

§ 371 Date: Jun. 13, 2022

(30) Foreign Application Priority Data
Dec. 13, 2019 (FR) FR1914356

(51) Int. Cl. A61M 21/00; G02B 27/10; G02B 27/12; B60Q 3/70; G01P 15/18

(52) U.S. Cl. CPC A61M 21/00; G02B 27/106; G02B 27/126; B60Q 3/70; G01P 15/18; A61M 2021/0044

(57) ABSTRACT

An anti-motion sickness device fitted to a motor vehicle including a triaxial accelerometer to detect the vehicle accelerations along three axes and to emit a corresponding acceleration signal; a display component of light markers to form at least one first and second artificial horizon lines at first and second inner surface, respectively of the motor vehicle. The device includes a control unit for receiving the acceleration signals and for driving the display wherein the artificial horizon lines are aligned in a horizontal plane perpendicular or substantially perpendicular to the gravity vector regardless of vehicle accelerations. The control unit is configured to process the acceleration signals, prior to the control of the display to determine in real time an acceleration frequency according to each of the accelerometer axes and to drive the display only when the acceleration frequency is below a threshold frequency, below which the motion sickness likely occurs.

### TECHNICAL FIELD

The disclosure concerns a device intended to combat the kinetosis integrated into a motor vehicle and a motor vehicle equipped with said device.

### BACKGROUND

A common problem for people traveling in a vehicle, plane or boat is motion sickness, also known as kinetosis. Motion sickness is caused by a mismatch between the sensations felt in the vestibular system of the internal ear and those experienced by other senses, such as a person's visual perceptions. Balance receptors in the internal ear are sensitive to gravity (for example, changes in orientation), speed, and changes in velocity (accelerations) that occur as the vehicle is displaced. When the sensations felt by the internal ear do not correspond to the visual signals perceived by the person, it often results in motion sickness for the person, manifesting in particular in the form of nausea and headaches.

For example, a passenger traveling on a winding road in an motor vehicle experiences linear and angular accelerations each time the vehicle is displaced through a curve. The vestibular sensing system's response to acceleration caused by vehicle motion will not correspond to visual perception unless the person is continuously looking at the road, so their internal ear's perception corresponds to the vehicle's path perceived visually through curves. It is for this reason that the driver of a vehicle does not usually suffer from motion sickness, while the passengers of the vehicle can suffer from it. Indeed, the driver constantly watches the road and visually perceives the movement of the vehicle so that the visual perceptions correspond to the senses of the internal ear. Conversely, the passengers of the vehicle who read or look only inside the vehicle or engage in other activities that prevent them from watching the road will have a visual perception that does not correspond to that of their internal ear.

To avoid motion sickness, one solution is therefore for the passenger to watch the road as if he were driving the vehicle, so that the visual information he receives corresponds more closely to the sensations of his vestibular system. However, when seated in the back of the vehicle, he usually partially sees the road. The actual movement of the passenger perceived by the internal auditory apparatus of the vestibular ear cannot therefore be easily associated with the visual perception of this movement. Motion sickness cannot therefore be avoided in this case.

Another solution to motion sickness, described in document US 2019/0083739, consists in using light markers in the internal uprights of a vehicle passenger compartment, said light markers forming light columns on each side of the passenger compartment. The height of the light columns is controlled by electronic control means so as to create an artificial horizon corresponding to the perception of the internal ear of a passenger seated in the vehicle. Nonetheless, this solution has the drawback of displaying the light markers irrespective of the displacement of the vehicle. Thus, in this known solution, an artificial horizon is constantly displayed in the passenger compartment. However, it is scientifically proven that the kinetosis only occurs for a very specific acceleration frequency range. In particular, the first warning symptoms of a kinetosis generally occur for acceleration frequencies lower than 25 Hz. The first signs of discomfort in passengers only really appear in the acceleration frequency ranges between 4 and 8 Hz. However, the kinetosis only becomes truly annoying for the passengers in the case where the accelerations in the front-rear or left-right direction of the vehicle have a frequency lower than 0.5 Hz or in the case where the accelerations in the vertical direction have a frequency lower than 2 Hz. Therefore, the solution described in document US 2019/0083739 has the disadvantage of proceeding with displaying light markers inside the vehicle even when the risk of kinetosis is zero or near-zero. This untimely display can therefore bother passengers if they are not yet suffering from kinetosis. Furthermore, the continuous use of light sources for the display of the light markers generates an overconsumption of electrical energy which, in particular in the case of an electric vehicle for example, can have a significant impact on the autonomy of the vehicle.

### SUMMARY

The disclosure therefore aims to provide a device intended to combat the kinetosis integrated into a motor vehicle and not having the drawbacks of the aforementioned prior art.

To this end, the disclosure concerns an anti-kinetosis device fitted to a motor vehicle, the anti-kinetosis device comprising:

a triaxial accelerometer configured to detect the accelerations of the vehicle along 3 axes and to emit a corresponding acceleration signal;

means for displaying light markers capable of forming at least one first artificial horizon line at the level of a first internal surface of the motor vehicle and at least one second artificial horizon line at the level of a second internal surface of the motor vehicle, said first and second artificial horizon lines being perpendicular or substantially perpendicular to each other;

a control unit capable of receiving the acceleration signals emitted by the accelerometer and of driving the display means so that the first and second artificial horizon lines are aligned in a horizontal plane perpendicular or substantially perpendicular to the gravitation vector, whatever the accelerations of the vehicle, the control unit being moreover configured to process said acceleration signals, prior to the control of the display means, so as to determine in real time an acceleration frequency along each of the axes of the accelerometer and to drive said display means only when said acceleration frequency is lower than a threshold frequency below which the kinetosis is liable to occur.

Thus configured, the anti-kinetosis device of the disclosure makes it possible to eliminate motion sickness by displaying two artificial horizon lines, respectively one in front of a person seated in the vehicle and the other beside said person, said lines being aligned in a horizontal plane. The device also makes it possible to display the light markers only for the acceleration frequency ranges where the risk of kinetosis is high.

The device of the disclosure may also comprise one or more of the following characteristics:

the threshold frequency is equal to 25 Hz.

the threshold frequency is between 4 and 8 Hz.

the threshold frequency is equal to 0.5 Hz for the accelerations along a longitudinal axis, corresponding to the front-rear direction of the vehicle, and along a lateral axis, corresponding to the right-left direction of the vehicle, and is equal to 2 Hz for the accelerations along a vertical axis, corresponding to the up-down direction of the vehicle.

the display means are capable of emitting at least two light beams of a rectilinear shape, respectively a first light beam projected onto the first internal surface and forming the first artificial horizon line, and a second light beam projected onto the second internal surface and forming the second artificial horizon line.

the display means comprise at least one light source emitting a main light beam, and means for separating and deflecting said main light beam into two secondary light beams.

the separation and deflection means comprise a prism intended to separate the light beam into two secondary light beams and a combination of mirrors and/or lenses intended to modify the path of said secondary light beams.

the display means comprise at least one pair of light sources, respectively a first light source emitting the first light beam and a second light source emitting the second light beam.

the light source, respectively the pair of light sources, is a laser, respectively a pair of lasers.

the display means comprise at least three light columns oriented vertically, respectively a first light column disposed in alignment with the first internal surface and in alignment with the second internal surface, a second light column disposed in alignment with the second internal surface and to the left or to the right of the first light column, and a third light column disposed in alignment with the first internal surface and closer to the rear of the vehicle than the first light column, each of the light columns being formed of a plurality of light points aligned in the vertical direction, each of the light points being able to emit light in an activated state and not emit light in a deactivated state, and in that the first artificial horizon line is formed by the virtual straight line passing through the highest or lowest activated light points on the first and third light columns respectively and the second artificial horizon line is formed by the virtual straight line passing through the highest, or lowest activated light points, on the first and second light columns respectively.

each of the light columns comprises a linear array of vertically aligned light-emitting diodes, each of the light-emitting diodes forming a light point.

The disclosure also concerns a motor vehicle equipped with the anti-kinetosis device as defined above.

In a particular configuration of the disclosure, the vehicle comprises at least one central upright, said central upright supporting the display means, said display means being configured to project the first light beam onto a rear lateral window or a rear door panel of the vehicle and to project the second light beam onto an internal surface of the passenger compartment disposed substantially perpendicular to said window or to said door panel, for example the backrest of one of the front seats of the vehicle.

The disclosure also concerns a method for displaying light markers intended to combat the kinetosis, comprising the following steps:

detecting by means of a triaxial accelerometer the accelerations of a vehicle along 3 axes and sending corresponding signals to a control unit;

determining in real time an acceleration frequency along each of the axes of the accelerometer and comparing the determined acceleration frequency and a threshold frequency below which the kinetosis is likely to occur;

in the case where the determined acceleration frequency is lower than the threshold frequency, driving by the control unit of means for displaying light markers capable of forming at least one first artificial horizon line at the level of a first internal surface of the motor vehicle and at least one second artificial horizon line at the level of a second internal surface of the motor vehicle, said first and second artificial horizon lines being perpendicular or substantially perpendicular to each other, such that the first and second artificial horizon lines are aligned in a horizontal plane perpendicular to the gravitation vector, whatever the accelerations of the vehicle.

### BRIEF DESCRIPTION OF THE DRAWINGS

Other features and advantages will become apparent from the description below of two particular embodiments of the disclosure, given by way of non-limiting examples, with reference to the appended drawings in which:

FIG. 1 is a perspective view of the passenger compartment of a vehicle according to a first embodiment of the disclosure;

FIG. 2 is a perspective view of the passenger compartment of a vehicle according to a second embodiment of the disclosure;

FIG. 3 is a rear view of a vehicle according to the disclosure, the vehicle being subjected to an acceleration along the Y axis;

FIG. 4 is a lateral view of a vehicle according to the disclosure, the vehicle being subjected to an acceleration along the X axis; and

FIG. 5 is a rear view of a vehicle according to the disclosure, the vehicle being subjected to an acceleration along the Y axis.

### DETAILED DESCRIPTION OF THE DRAWINGS

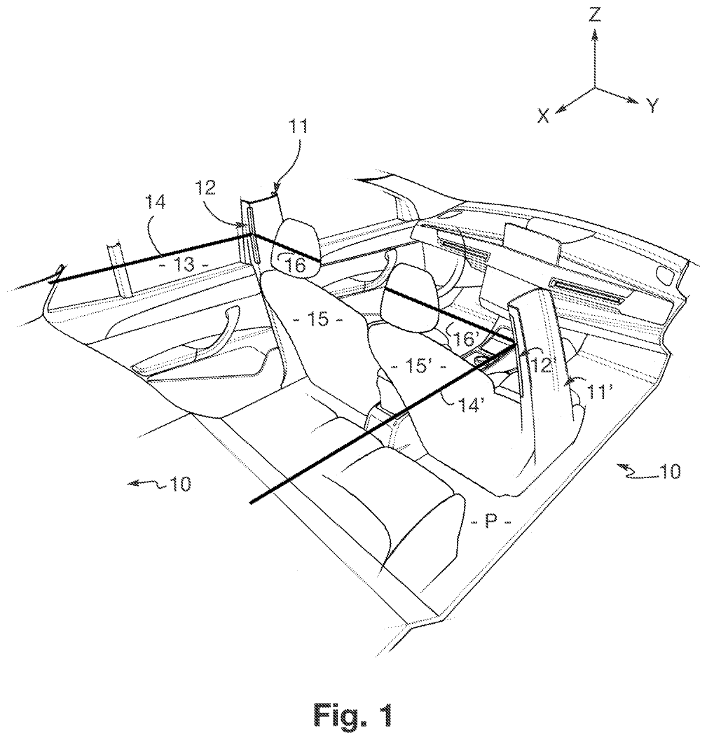

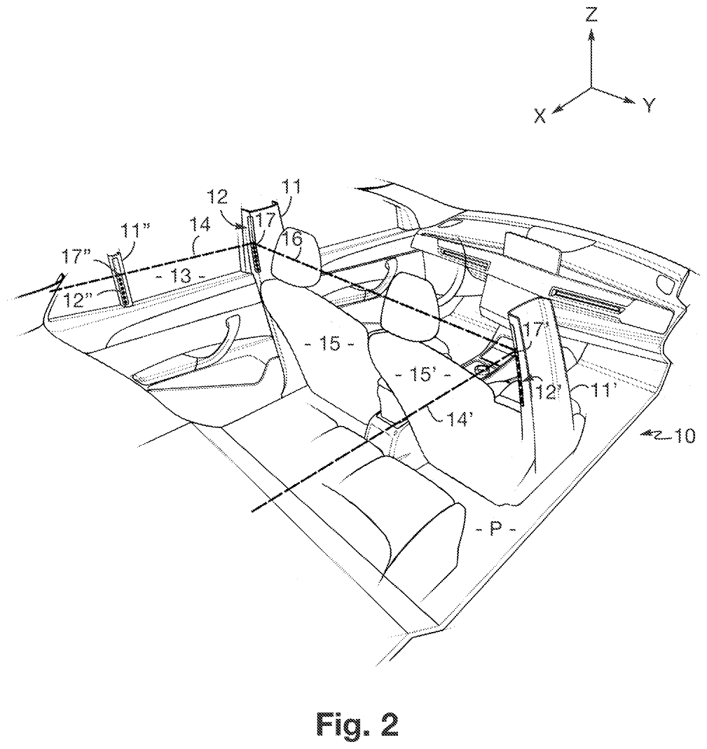

In the remainder of this description, and with reference to the Cartesian coordinate system XYZ shown in FIGS. 1 and 2, the terms “longitudinal direction” will be used for a direction along the X axis, “transverse direction” for a direction along the Y axis and “vertical direction” for a direction along the Z axis. Moreover, conventionally, the term “front” will be used to indicate an orientation directed towards the front of the vehicle and the term “rear” will be used to indicate an orientation directed towards the rear of the vehicle.

With reference to FIG. 1, there is shown a passenger compartment of a vehicle 10 according to a first embodiment of the disclosure. In this embodiment, a first lighting column 12 oriented vertically is supported by a central upright 11 of the vehicle and is disposed on the left side with respect to the field of vision of a passenger seated in the back of the vehicle, and a second lighting column 12′ oriented vertically is supported by another central upright 11′ of the vehicle and is disposed on the right side with respect to the field of vision of a passenger seated in the back of the vehicle. Each lighting column 12, 12′ comprises at least one light source configured to project a first rectilinear light beam onto a rear lateral window or a rear door panel 13 of the vehicle and to project a second rectilinear light beam onto the backrest of one of the front seats 15 or 15′ of the vehicle. The first light beam emitted by the first lighting column 12 projects onto the rear lateral window or the rear door panel 13 directly adjacent to the first lighting column 12 along a first line 14, subsequently referenced as the first artificial horizon line, and the second light beam emitted by the first lighting column 12 is projected onto the backrest of the front seat 15 directly adjacent to the first lighting column 12 along a second line 16, subsequently referenced as the second artificial horizon line. Similarly, the first light beam emitted by the second lighting column 12′ projects onto the rear lateral window or the rear door panel (not shown) directly adjacent to the second lighting column 12′ along a first line 14′, subsequently referenced as the first artificial horizon line, and the second light beam emitted by the second lighting column 12′ projects onto the backrest of the front seat 15′ directly adjacent to the second lighting column 12′ along a second line 16′, subsequently referenced as the second artificial horizon line. In this configuration, the first and second artificial horizon lines are perpendicular or substantially perpendicular to each other.

By suitably driving the light source(s), it is thus possible to align the first and second artificial horizon lines 14, 16 and 14′, 16′ in a horizontal plane which is always perpendicular to the gravitation vector. Thus, a person seated next to the rear lateral window 13 and behind the front seat 15 and fixing said artificial horizon lines 14, 16 will have the same visual sensations as the driver looking at the road: he will therefore no longer be subject to motion sickness. To achieve this result, the vehicle 10 is advantageously equipped with a triaxial accelerometer configured to detect the accelerations of the vehicle along the 3 axes X, Y and Z and to emit corresponding acceleration signals, and a control unit capable of receiving the acceleration signals emitted by the accelerometer and of driving the light source(s) so that the first and second artificial horizon lines 14, 16 and 14′, 16′ are aligned in a horizontal plane A, perpendicular to the gravitation vector, whatever the accelerations of the vehicle, the control unit being moreover configured to process said acceleration signals, prior to the control of the light sources, so as to determine in real time an acceleration frequency along each of the axes of the accelerometer and to drive said light sources only when said acceleration frequency is lower than a threshold frequency below which the kinetosis is likely to occur.

With reference to FIG. 2, there is shown a passenger compartment of a vehicle according to a second embodiment of the disclosure. In this embodiment, the anti-kinetosis device comprises at least three light columns oriented vertically, respectively a first light column 12 which is supported by a central upright 11 of the vehicle and which is disposed on the left side with respect to the field of vision of a passenger seated in the back of the vehicle, a second light column 12′ which is supported by another central upright 11′ of the vehicle and which is disposed on the right side with respect to the field of vision of a passenger seated in the back of the vehicle, and a third light column 12″ which is supported by a lateral upright 11″ forming part of a rear lateral window or a rear door panel 13 and on the left side with respect to the field of vision of a passenger seated in the back of the vehicle. Each light column 12, 12′, 12″ is formed of a plurality of light points aligned in the vertical direction, each of the light points being able to emit light in an activated state and not emit light in a deactivated state. Thus, a first artificial horizon line 14 is formed by the virtual straight line passing through the highest activated light points 17 and 17″ on the first and third light columns 12, 12″ respectively, and a second artificial horizon line 16 is formed by the virtual straight line passing through the highest activated light points 17 and 17′ on the first and second light columns 12, 12′ respectively. In this configuration, the first and second artificial horizon lines 14, 16 are perpendicular or substantially perpendicular to each other. In an advantageous variant of the disclosure, each of the light columns 12, 12′ and 12″ may comprise, for example, a linear array of vertically aligned light-emitting diodes, each of the light-emitting diodes forming one of the light points of the light columns. In another configuration of the disclosure, the light points can be activated starting from the top of each light column. In this case, the first artificial horizon line 14 will be formed by the virtual straight line passing through the lowest activated light points on the first and third light columns 12, 12″ respectively, and the second artificial horizon line 16 will be formed by the virtual straight line passing through the lowest activated light points on the first and second light columns 12, 12′ respectively. Moreover, the vehicle 10 may advantageously comprise a fourth light column (not shown) which will be supported by a lateral upright forming part of a rear lateral window or of a rear door panel and on the right side with respect to the field of vision of a passenger seated in the back of the vehicle, said fourth light column making it possible to define, in combination with the second light column 12′, a third artificial horizon line 14′ on the right side of the vehicle.

By suitably driving the light-emitting diodes of the first, second and third light columns 12, 12′ and 12″, it is thus possible to align the first and second artificial horizon lines 14, 16 in a horizontal plane which is always perpendicular to the gravitation vector. Thus, a person seated next to a rear lateral window or a rear lateral door panel 13 and behind the front seat 15 and fixing said artificial horizon lines 14, 16 will have the same visual sensations as the driver looking at the road: he will therefore no longer be subject to motion sickness. The control unit will also be configured to process said acceleration signals, prior to the drive of the light-emitting diodes, so as to determine in real time an acceleration frequency along each of the axes of the accelerometer and to drive said light-emitting diodes only when said acceleration frequency is lower than a threshold frequency below which the kinetosis is likely to occur.

The two embodiments described above are obviously not limiting for the disclosure. Other embodiments could be envisaged at this level.

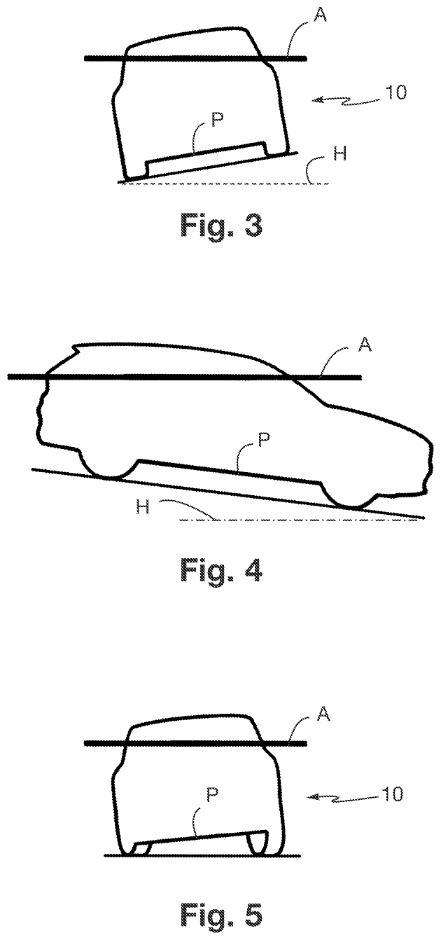

FIGS. 3 to 5 illustrate several possible driving conditions and the corresponding operation of the anti-kinetosis device of the disclosure.

Thus, in the case where the vehicle 10 is traveling on a road inclined to the left with respect to a fixed horizontal plane H, perpendicular to the gravitation vector, as shown in FIG. 3, it is subjected to an acceleration along the Y axis. This acceleration is detected by the triaxial accelerometer, which sends a corresponding acceleration signal to the control unit. In response to this acceleration signal, the control unit determines in real time an acceleration frequency along each of the axes of the accelerometer and compares this acceleration frequency with a threshold frequency below which the kinetosis is likely to occur. In the present case, the acceleration frequency determined in real time along the Y axis being lower than the threshold frequency, the control unit controls the display of the light markers intended to form the artificial horizon lines in such a way that they are aligned in a horizontal plane A inclined with respect to the floor P of the vehicle at the level of the Y axis.

In the case where the vehicle 10 is traveling on a road inclined downwards with respect to a fixed horizontal plane H, perpendicular to the gravitation vector, as shown in FIG. 4, it is subjected to an acceleration along the X axis. This acceleration is detected by the triaxial accelerometer, which sends a corresponding acceleration signal to the control unit. In response to this acceleration signal, the control unit determines in real time an acceleration frequency along each of the axes of the accelerometer and compares this acceleration frequency with a threshold frequency below which the kinetosis is likely to occur. In the present case, the acceleration frequency determined in real time along the X axis being lower than the threshold frequency, the control unit controls the display of the light markers intended to form the artificial horizon lines in such a way that they are aligned in a horizontal plane A inclined with respect to the floor P of the vehicle at the level of the X axis.

In the case where the vehicle 10 is travelling on a flat road and makes a left turn, as shown in FIG. 5, it is subjected to an acceleration along the Y axis. This acceleration is detected by the triaxial accelerometer, which sends a corresponding acceleration signal to the control unit. In response to this acceleration signal, the control unit determines in real time an acceleration frequency along each of the axes of the accelerometer and compares this acceleration frequency with a threshold frequency below which the kinetosis is likely to occur. In the present case, the acceleration frequency determined in real time along the Y axis being lower than the threshold frequency, the control unit controls the display of the light markers intended to form the artificial horizon lines in such a way that they are aligned in a horizontal plane A inclined with respect to the floor P of the vehicle at the level of the Y axis.

Moreover, the disclosure also aims to protect a method for displaying light markers intended to combat the kinetosis. This method may in particular comprise the following steps:

detecting by means of a triaxial accelerometer the accelerations of a vehicle along 3 axes and sending corresponding signals to a control unit;

determining in real time an acceleration frequency along each of the axes of the accelerometer and comparing the determined acceleration frequency and a threshold frequency below which the kinetosis is likely to occur;

in the case where the determined acceleration frequency is lower than the threshold frequency, driving by the control unit of means for displaying light markers capable of forming at least one first artificial horizon line at the level a first internal surface of the motor vehicle and at least one second artificial horizon line at the level of a second internal surface of the motor vehicle, said first and second artificial horizon lines being perpendicular or substantially perpendicular to each other, such that the first and second artificial horizon lines are aligned in a horizontal plane perpendicular to the gravitation vector, whatever the accelerations of the vehicle.

1. An anti-kinetosis device fitted to a motor vehicle, the anti-kinetosis device comprises:
a triaxial accelerometer configured to detect the accelerations of the vehicle along 3 axes and to emit a corresponding acceleration signal;
means for displaying light markers capable of forming at least one first artificial horizon line at the level of a first internal surface of the motor vehicle and at least one second artificial horizon line at the level of a second internal surface of the motor vehicle, said first and second artificial horizon lines being perpendicular or substantially perpendicular to each other; and
a control unit capable of receiving the acceleration signals emitted by the accelerometer and of driving the display means so that the first and second artificial horizon lines are aligned in a horizontal plane, perpendicular or substantially perpendicular to the gravitation vector, whatever the accelerations of the vehicle, the control unit being moreover configured to process said acceleration signals, prior to the control of the display means, so as to determine in real time an acceleration frequency along each of the axes of the accelerometer and to drive said display means only when said acceleration frequency is lower than a threshold frequency below which the kinetosis is likely to occur.

2. The device according to claim 1, wherein the threshold frequency is equal to 25 Hz.

3. The device according to claim 1, wherein the threshold frequency is between 4 and 8 Hz.

4. The device according to claim 1, wherein the threshold frequency is equal to 0.5 Hz for the accelerations along a longitudinal axis, corresponding to the front-rear direction of the vehicle, and along a lateral axis, corresponding to the right-left direction of the vehicle, and is equal to 2 Hz for accelerations along a vertical axis, corresponding to the up-down direction of the vehicle.

5. The device according to claim 1, wherein the display means are capable of emitting at least two light beams of a rectilinear shape, respectively a first light beam projected on the first internal surface and forming the first artificial horizon line, and a second light beam projected onto the second internal surface and forming the second artificial horizon line.

6. The device according to claim 5, wherein the display means comprise at least one light source emitting a main light beam, and means for separating and deflecting said main light beam into two secondary light beams.

7. The device according to claim 6, wherein the separation and deflection means comprise a prism intended to separate the light beam into two secondary light beams and a combination of mirrors and/or lenses intended to modify the path of said secondary light beams.

8. The device according to claim 5, wherein the display means comprise at least one pair of light sources, respectively a first light source emitting the first light beam and a second light source emitting the second light beam.

9. The device according to claim 6, wherein the light source, respectively the pair of light sources, is a laser, respectively a pair of lasers.

10. The device according to claim 1, wherein the display means comprise at least three light columns oriented vertically, respectively a first light column disposed in alignment with the first internal surface and in alignment with the second internal surface, a second light column disposed in alignment with the second internal surface and to the left or to the right of the first light column, and a third light column disposed in alignment with the first internal surface and closer to the rear of the vehicle than the first light column, each of the light columns, being formed of a plurality of vertically aligned light points, each of the light points being able to emit light in an activated state and not emit light in a deactivated state, and in that the first artificial horizon line is formed by the virtual straight line passing through the highest or lowest activated light points on the first and third light columns respectively and the second artificial horizon line is formed by the virtual straight line passing through the highest or lowest activated light points on the first and second light columns respectively.

11. The device according to claim 10, wherein each of the light columns comprises a linear array of vertically aligned light-emitting diodes, each of the light-emitting diodes forming a light point.

12. A motor vehicle equipped with an anti-kinetosis device according to claim 1.

13. The motor vehicle equipped with an anti-kinetosis device according to claim 5, wherein it comprises at least one central upright, said central upright supporting the display means, said display means being configured to project the first light beam onto a rear lateral window or rear door panel of the vehicle and to project the second light beam onto an internal surface of the passenger compartment disposed substantially perpendicular to said window or to said door panel, for example the backrest of one of the front seats of the vehicle.

14. A method for displaying light markers intended to combat the kinetosis, comprising the following steps:
detecting by means of a triaxial accelerometer the accelerations of a vehicle along 3 axes and sending corresponding signals to a control unit;
determining in real time an acceleration frequency along each of the axes of the accelerometer and comparing the determined acceleration frequency and a threshold frequency below which the kinetosis is likely to occur;
in the case where the determined acceleration frequency is lower than the threshold frequency, driving by the control unit of means for displaying light markers capable of forming at least one first artificial horizon line at the level of a first internal surface of the motor vehicle and at least one second artificial horizon line at the level of a second internal surface of the motor vehicle, said first and second artificial horizon lines being perpendicular or substantially perpendicular to each other, such that the first and second artificial horizon lines are aligned in a horizontal plane, perpendicular to the gravitation vector, whatever the accelerations of the vehicle.
